(19) United States
(12) Patent Application Publication
(10) Pub. No.: US 2025/0208108 A1
(43) Pub. Date: Jun. 26, 2025

(54) STEM RESPIRATION SYSTEM FOR PLANTS

(71) Applicant: The Regents of the University of California, Oakland, CA (US)

(72) Inventor: Kolby Jardine, Berkeley, CA (US)

(21) Appl. No.: 18/982,578

(22) Filed: Dec. 16, 2024

Related U.S. Application Data
(60) Provisional application No. 63/613,147, filed on Dec. 21, 2023.

(51) Int. Cl. G01N 33/00; G01N 21/3504

(52) U.S. Cl. CPC G01N 33/0098; G01N 21/3504

(57) ABSTRACT

This disclosure provides systems, methods, and apparatus related to stem respiration in plants. In one aspect, a system includes a chamber, a first carbon dioxide sensor, and a pump. The chamber includes an inlet port and an outlet port. The chamber forming an enclosed volume when attached to a stem of a plant. The first carbon dioxide sensor includes a first sensor inlet connected to the outlet port of the chamber. The pump is connected to a first sensor outlet of the first carbon dioxide sensor. The pump operable is to draw the air though the inlet port of the chamber and through the first carbon dioxide sensor.

### RELATED APPLICATIONS

This application claims priority to U.S. Provisional Patent Application No. 63/613,147, filed Dec. 21, 2023, which is herein incorporated by reference.

### STATEMENT OF GOVERNMENT SUPPORT

This invention was made with government support under Contract No. DE-AC02-05CH11231 awarded by the U.S. Department of Energy. The government has certain rights in this invention.

### BACKGROUND

Autotrophic aerobic respiration is the controlled oxidation of photosynthetically fixed carbon by plants resulting in the consumption of molecular oxygen (O₂) and the production of carbon dioxide (CO₂). In non-photosynthetic tissues, aerobic respiration is a major cellular source of usable chemical energy (adenosine triphosphate (ATP)), reducing power (nicotinamide adenine dinucleotide (NAD)+hydrogen (NADH)), and source of carbon skeletons. Carbon skeletons are needed in numerous physiological processes including maintenance of existing tissues, growth and development, reproduction, defensive and signaling processes during responses to abiotic and biotic stress, and senescence processes. Despite the high rates of CO₂ photo-assimilation in leaves, aerobic respiration in all plant tissues (and photorespiration in leaves during the day) leads to a large fraction of assimilated carbon returning to the atmosphere as CO₂. While highly uncertain, autotrophic respiration of terrestrial ecosystems represents a major atmospheric source of CO₂, with an annual global source estimated between 4 to 7 times that of anthropogenic fossil fuel combustion.

In dynamic vegetation models, autotrophic respiration is often calculated as the sum of leaf, stem, and root respiration. While environmental and biological influences over leaf respiration during the day and night are becoming increasingly common across biomes globally due to the availability of numerous commercial dynamic leaf gas exchange systems, limited observations of dynamic stem gas exchange have been reported, likely constrained by a lack of commercial sensors. Respired CO₂ in tree stems can diffuse to the atmosphere driven by the concentration gradient between the inner bark and ambient air. This mechanism is known as stem CO₂ efflux (Eₛ, μmol m⁻² s⁻¹) and is estimated to represent a large but uncertain fraction of total autotrophic respiration of trees.

### SUMMARY

One innovative aspect of the subject matter described in this disclosure can be implemented in a system including a chamber, a first carbon dioxide sensor, and a pump. The chamber includes an inlet port and an outlet port. The chamber forms an enclosed volume when attached to a stem of a plant. The first carbon dioxide sensor includes a first sensor inlet connected to the outlet port of the chamber. The pump is connected to a first sensor outlet of the first carbon dioxide sensor. The pump is operable to draw the air though the inlet port of the chamber and through the first carbon dioxide sensor.

Another innovative aspect of the subject matter described in this disclosure can be implemented in a system including a chamber, a first carbon dioxide sensor, a second carbon dioxide sensor, and a pump. The chamber includes an inlet port and an outlet port. The chamber forms an enclosed volume when attached to a stem of a plant. The first carbon dioxide sensor includes a first sensor inlet connected to the outlet port of the chamber. The second carbon dioxide sensor includes a second sensor inlet. The second sensor inlet operable to sample ambient air. The pump is connected to a first sensor outlet of the first carbon dioxide sensor and a second sensor outlet of the second carbon dioxide sensor. The pump is operable to draw the air though the inlet port of the chamber and through the first carbon dioxide sensor. The pump is operable to draw the ambient air though the second sensor inlet and through the second carbon dioxide sensor.

Details of one or more embodiments of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1A shows an example of a schematic illustration of a stem respiration system. FIG. 1B shows an example of a schematic illustration of a specific implementation of stem respiration system.

FIG. 2A shows an example of a schematic illustration of a stem chamber. FIG. 2B shows an example of a schematic illustration of a stem chamber positioned on a tree.

FIG. 3A shows an example of diurnal CO₂ concentrations in ambient air and stem chamber air. FIG. 3B shows an example of diurnal CO₂ stem Eₛ flux together with air and stem temperature from an Ash tree (at breast height) in Fort Collins, CO, USA.

FIG. 4 shows an example of a scatter plot and linear regression between Eₛ flux and air temperature from an Ash tree (at breast height) in Fort Collins, CO, USA.

### DETAILED DESCRIPTION

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise.

The terms “about” or “approximate” and the like are synonymous and are used to indicate that the value modified by the term has an understood range associated with it, where the range can be ±20%, ±15%, ±10%, ±5%, or ±1%. The terms “substantially” and the like are used to indicate that a value is close to a targeted value, where close can mean, for example, the value is within 80% of the targeted value, within 85% of the targeted value, within 90% of the targeted value, within 95% of the targeted value, or within 99% of the targeted value.

Limited studies, most of which have employed a commercial system designed for soil respiration and adapted to stems, have utilized the static technique to estimate stem CO₂ efflux (Eₛ). For this method, air inside a stem chamber is recirculated through an Infrared Gas Analyzer (IRGA) for CO₂ concentration measurements. The rate of CO₂ accumulation over time is then used to estimate Eₛ. This static method is primarily adapted to stems from the use of existing commercial soil respiration systems. Environmental variables impacting soil respiration within forested ecosystems are generally considered to change slowly throughout the day, with air pressure control to minimize pressure related artifacts deemed more important than fast and continuous flux measurements.

While having the advantage of simplicity due to the need for only a single IRGA for CO₂, the static method suffers from numerous issues that limit its potential value as a tool in dynamic Eₛ studies and the influence of biological and environmental variables. As Eₛ is not directly measured, but instead estimated from the slope of CO₂ concentration versus time, very high CO₂ concentrations (thousands of ppm CO₂) rapidly build up inside the enclosure, reducing the CO₂ concentration gradient between the inner bark and ambient air. This in turn reduces the CO₂ efflux and can therefore lead to underestimates of CO₂ efflux rates. Moreover, the method assumes a leak free enclosure, where ambient air is prevented from entering the chamber by sealing the chamber to the stem with various glues. However, small leaks, which are difficult to detect and quantify in the field, reduce the rate at which CO₂ accumulates in the stem chamber, and quickly become more significant with time as the CO₂ concentration inside the chamber rapidly increases above ambient air levels. Moreover, after each measurement period lasting 5 minutes to 30 minutes, the stem enclosure is removed from the stem to reintroduce ambient air. Alternatively, the chambers must be rapidly flushed with ambient air just prior to each Eₛ measurement, increasing the complexity.

Thus, stem respiration measurements using the static method typically require manual installation and deinstallation for each measurement point. This leads to poor time resolution, making the method generally unable to resolve potentially large diurnal patterns in Eₛ as well as fast dynamics on the time scales of <15 minutes associated changes in sap velocity and incoming sunlight during the passing of clouds, for example.

In addition, high humidity environments are often encountered near the base of trees where most stem Eₛ observations have been reported, with stem transpiration often leading to significant condensation inside the CO₂ IRGAs. As IRGAs do not function under saturating humidity conditions, a complete loss of data is often encountered when condensation occurs, especially if Eₛ measurements are sequentially performed over time.

In summary, static chambers suffer from a number of issues including high humidity and condensation issues, require a rigorously leak-free enclosure which is difficult to verify in the field, quickly generate a greatly altered CO₂ stem atmosphere that can lead to errors in determining Eₛ by greatly altering stem-atmosphere concentration gradients, and the requirement to flush the enclosure with ambient air before starting each measurement, increasing complexity and constraining the time resolution of Eₛ observations.

The lack of a low-cost commercially available system for monitoring real-time stem Eₛ under challenging field conditions precludes a comprehensive analysis of the dependence of diurnal stem Eₛ on biophysical (e.g., wood density, sap wood volume, bark thickness), physiological (e.g., growth, net photosynthesis, transpiration, and aerobic respiration rates), biochemical (e.g., volatile organic compound metabolism, nutrient and respiratory substrates and pathways), and environmental (e.g., temperature, light, moisture availability) factors.

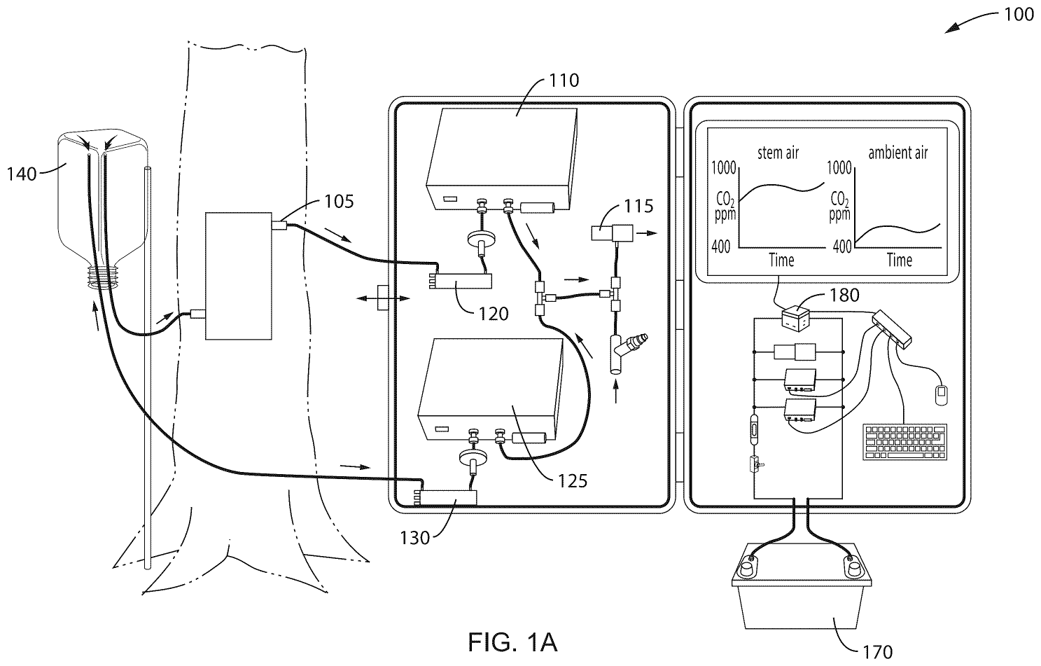

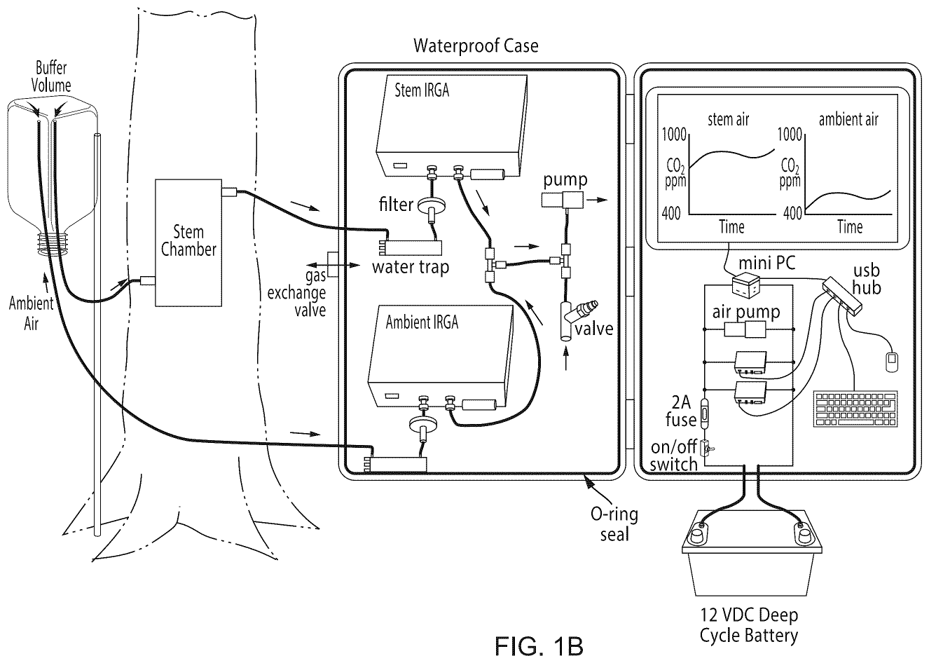

Described herein is a system for real-time monitoring of stem Eₛ. FIG. 1A shows an example of a schematic illustration of a stem respiration system. FIG. 1B shows an example of a schematic illustration of a specific implementation of stem respiration system.

Starting with FIG. 1A, a system includes a chamber 105, a first carbon dioxide sensor 110, and a pump 115. The chamber 105 may also be referred to as a stem chamber herein. The chamber 105 includes an inlet port and an outlet port. The chamber 105 forms an enclosed volume when attached to a stem of a plant (e.g., a tree). The volume formed by the chamber 105 and the stem of a plant is enclosed except for the inlet port and the outlet port of the chamber 105. The first carbon dioxide sensor 110 includes a first sensor inlet connected to the outlet port of the chamber 105. The pump 115 is connected to a first sensor outlet of the first carbon dioxide sensor 110. The pump 115 is operable to draw the air though the inlet port of the chamber 105 and through the first carbon dioxide sensor 110. The air from the inlet port of the chamber 105 flows by the stem of the plant. The air that flows through the first carbon dioxide sensor 110 includes any carbon dioxide generated by the area of the stem of the plant forming the enclosed volume.

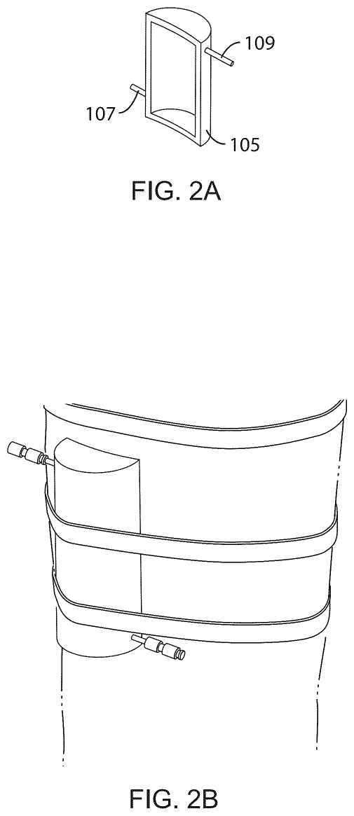

FIG. 2A shows an example of a schematic illustration of a stem chamber. As shown in FIG. 2A, the chamber 105 includes the inlet port 107 and the outlet port 109. FIG. 2B shows an example of a schematic illustration of a stem chamber positioned on a tree.

In some embodiments, the chamber 105 comprises a plastic. In some embodiments, the chamber 105 comprises polyethylene terephthalate glycol (PEG). In some embodiments, the enclosed volume formed by the chamber 105 and the stem of the plant is about 200 milliliters (mL) to 400 mL. In some embodiments, an area on the stem of the plant forming the enclosed volume is about 90 cm² to 144 cm². In some embodiments, an area on the stem of the plant forming the enclosed volume is a rectangular or square area. In some embodiments, dimensions of an area on the stem of the plant forming the enclosed volume are about 15 centimeters (cm) to 18 cm by about 6 cm to 8 cm.

In some embodiments, the chamber 105 has a shape of a portion of a cylinder (e.g., a cylindrical section made by cutting a hollow cylinder along an axis perpendicular to the radius of the cylinder). In some embodiments, the inlet port 107 is proximate a bottom end of the chamber 105 (e.g., in the curved surface of the chamber 105 when the chamber is a cylindrical section). In some embodiment, the outlet port 109 is proximate a top end of the chamber 105 (e.g., in the curved surface of the chamber 105 when the chamber is a cylindrical section). With the inlet port 107 and the outlet port 109 of the chamber 105 positioned in this manner, the ambient air flows past the area of the stem that forms the enclosed volume. Further, having the inlet port 107 and the outlet port 109 of the chamber 105 positioned in this manner helps to prevent water (e.g., rain water that may collect in the chamber 105 over time chamber when the system is deployed in a rainy environment) from being pulled into the first carbon dioxide sensor 110.

In some embodiments, foam (not shown) is position between the chamber 105 and the stem of the plant. In some embodiments, the foam aids in creating or creates a seal between the chamber 105 and the stem of the plant. In some embodiments, the seal is an airtight seal. In some embodiments, the foam is a high density foam. In some embodiments, the foam is a high density polymer foam. In some embodiments, the foam is a high density polyurethane foam.

In some embodiments, the carbon dioxide sensor 110 comprises a carbon dioxide infrared gas analyzer.

In some embodiments, the system 100 further comprises a power source 170 operable to power the first carbon dioxide sensor 110 and the pump 115. In some embodiments, the power source 170 consists of or comprises a battery.

In some embodiments, the system 100 includes a computing device 180 operable to record carbon dioxide concentration information output from the first carbon dioxide sensor 110.

In some embodiments, the system 100 further comprises a first water trap 120 positioned between the chamber 105 and the first carbon dioxide sensor 110. The first water trap 120 is operable to remove water vapor from the air. Removing water vapor from the air prevents or aids from preventing condensation in the first carbon dioxide sensor 110. In some embodiments, the first water trap 120 comprises a membrane dryer. In some embodiments, the first water trap 120 includes clay granules and dolomite.

In some embodiments, the system 100 further comprises a second carbon dioxide sensor 125. The second carbon dioxide sensor 125 is used to measure the CO₂ concentration in ambient air. Measuring the CO₂ concentration in ambient air allows any measured difference between the ambient air and the stem CO₂ concentrations to be attributed to respiratory activities of the stem.

The second carbon dioxide sensor 125 includes a second sensor inlet. The second sensor inlet operable to sample ambient air. The pump 115 is connected to a second sensor outlet of the second carbon dioxide sensor 125. The pump 115 is further operable to draw the ambient air though the second sensor inlet and through the second carbon dioxide sensor 125. In some embodiments, the second sensor inlet is connected to a tube that is positioned to sample ambient air.

In some embodiments, the second carbon dioxide sensor 125 comprises a carbon dioxide infrared gas analyzer.

In some embodiments, the system 100 further comprises a second water trap 130 positioned prior to the second sensor inlet of the second carbon dioxide sensor 125. The second trap 130 is operable to remove water vapor from the ambient air. In some embodiments, the computing device 180 is further operable to record carbon dioxide concentration information output from the second carbon dioxide sensor 125.

In some embodiments, the inlet ports of both the chamber 105 and the second sensor inlet of the second carbon dioxide sensor 125 are connected to tubes with the inlets of the tubes being positioned in a buffer volume 140. In some embodiments, the buffer volume 140 is a container with a single opening through which the tubes pass and through which ambient air also passes. The buffer volume 140 may be similar to a 2 liter plastic soda bottle or a 1 gallon plastic milk jug. In some embodiments, the buffer volume has a volume of about 1 liter (L) to 5 L, about 1 L, or about 5 L.

In some embodiments, the buffer volume 140 helps to prevent water from being pulled into the system 100. A buffer volume 140 may be used for this purpose even when the system 100 does not include the second carbon dioxide sensor 125.

In some embodiments, the buffer volume 140 helps to ensure that the chamber 105 and the second carbon dioxide sensor 125 are receiving the same ambient air. This helps to ensure the accuracy of the measurements. Further, the buffer volume 140 can smooth out transients in the CO₂ concentration in the ambient air. Such transients may be caused by a gasoline powered motor vehicle passing near the system 100.

In some embodiments, the system 100 includes two or more chambers 105 (i.e., a plurality of chambers). In some embodiments, when the system 100 includes two or more chambers, the system includes a valve (e.g. a continuous flow valve) such that ambient air is always being pulled into each of the chambers 105, but the first carbon dioxide sensor 110 only receives air from one of the chambers 105. In some embodiments, when the system 100 includes two or more chambers, the system includes a mass flow controller (not shown) such that ambient air is always being pulled into each of the chambers 105 at a constant rate.

With such a system 100, chambers 105 could be installed on different trees positioned near one another and the system 100 used to measure the carbon dioxide concentration from the different trees. For example, a carbon dioxide concentration from a first chamber 105 could be measured, the valve set to sample a second chamber 105, and then the carbon dioxide concentration from a second chamber 105 could be measured.

Further, with such a system 100, multiple chambers 105 could be positioned on the same tree, but at different heights along the stem of the tree. This would allow a researcher to determine the change in the respiration rate of the tree with height, if any, and also to measure the respiration rate of the entire tree or stem of the tree.

The following examples are intended to be examples of the embodiments disclosed herein, and are not intended to be limiting.

### Example—Design, Installation, and Operation of the Portable Stem Respiration System

Described below is a low cost, lightweight, waterproof system with low power requirements (about 0.1 A to 0.2 A at about 12 V) for real-time monitoring of stem Eₛ using a 3D printed dynamic stem chamber, dual IRGAs for continuous ambient and stem air CO₂ concentration observations, and a car battery. The system implements accurate and constant flow control through the chamber, continuous water removal, and CO₂ measurements of both the reference and stem enclosure air using two distinct IRGAs that are regularly “matched” such that any measured difference between the ambient air and stem CO₂ concentrations (Δ CO₂) can be attributed to respiratory activities of the stem. The design offers control over the flow rate through the stem chamber, minimizes complexity by eliminating the need for a pump to introduce air into the chamber, and reduces or eliminates water condensation issues by removing H₂O vapor prior to CO₂ analysis.

Following a calibration procedure with a 400 ppm CO₂ standard at the beginning of each weekly measurement campaign, the system showed low IRGA drift over time (Δ CO₂<10 ppm) and was highly sensitive to stem CO₂ efflux (observed Δ CO₂ ranging from 60-1,000 ppm). The system was used in Colorado to quantify Eₛ for 24 hours and the resulting flux was related to air temperature.

The main components used in the construction of the portable stem respiration system are listed below:
water proof case
carbon dioxide gas analyzer
1 micron filter assembly
water vapor scrub tube assembly
air pump
VDC mini-pc
minicomputer with battery powered portable monitor
3D printed stem chamber
gas flow meter

A 3D stem chamber (polyethylene terephthalate glycol, PEG) was printed using a 3D printer. To create a seal between the stem chamber base and the stem, an about ½″ thick rectangular foam rectangle was cut to the interior dimensions and glued to the inside base of the stem chamber using silicon sealant. ¼″ quick connect union fittings were then attached onto the stem ¼″ inlet and outlet port for quick connections to tubing. Following lightly cleaning the surface of the stem to be measured with a brush one day prior to measurements, the stem chamber was placed with the foam gaskets towards the stem and was secured using two cinch straps (see FIG. 2B). Adjacent to the tree and installed in the inverted position with the mouth at the same height as the stem chamber, an inverted 10 gallon ambient air buffer was installed on a vertical support structure.

All other items were installed and configured in a waterproof and breathable case with an integrated gas-exchange valve to equilibrate air pressure inside and outside of the case. A monitor was mounted to the inside lid. The electrical components, pump, fittings, water vapor traps, particle filters, CO₂ IRGAs, and gas sample tubing and fittings were installed inside of the case on top of the bottom foam layer. Power was supplied externally using a 12 VDC battery and distributed to the PC, pump, and two CO₂ IRGAs inside the case using a parallel circuit. In addition to the integral 2 A fast blow glass fuses protecting each of the CO₂ IRGAs internally, the 12 VDC circuit was protected from an overcurrent with a 5 A fast blow glass fuse.

To prepare the system for operation, the case is first opened, and fresh water absorbent is placed in the two water vapor traps which are then resealed. Following this, the two ¼″ caps on the outside of the case protecting the ambient and stem air inlets are removed and connected to the appropriate length of ¼″ sample tubing to reach from (1) the air inlet on the case to the stem chamber air outlet and (2) from the ambient air inlet on the case to inside the ambient air buffer. Note, keeping both tubing segments the same length helps to ensure that a similar air flow rate is established through the stem and ambient IRGAs. Following this, the power to the main unit is switched on, which automatically turns on the air sample pump and the two IRGAs. The mini-PC is then switched on and communication is established with the ambient air and stem air IRGAs via USB communication cables. The air flow rate entering the ambient air and stem air ¼″ sample tubing is then measured using a 0-500 mL/min flow meter. The air flow rate is adjusted through both ambient and stem air tubing together using the manual valve just upstream of the pump. Opening this valve decreases the flow rate through the IRGAs while closing this valve increases it. The valve is adjusted such that an about 80 ml/min to 100 ml/min is maintained through both ambient and stem IRGAs. The valve is then locked to ensure the flow is held constant throughout the duration of the stem respiration experiment (e.g., 24 hours).

### Example—Match and Calibration Procedure and Stem CO₂ Efflux Measurements

Once the specified flow rates are achieved, the delay time for each of the IRGAs should be separately determined by briefly blowing near the ambient air sample and stem tubing and recording the time required to observe the peak in CO₂ concentration on the monitor. Note that the delay with about 100 ml/min air flow through each of the sample tubes was determined to be less than 3 min due to the dead volume of the system, mainly the water trap.

The stem respiration system is then calibrated and matched prior to installation onto a tree and logging CO₂ concentrations on the mini-PC. The calibration and match procedure can be performed in the lab or field using a 10 L gas sample bag with 400 ppm CO₂. A ¼″ stainless steel tee fitting was used to connect both the ambient air and stem air to the opened bag containing the 400-ppm standard. Note that if both ambient and stem air IRGAs are flowing at 100 ml/min (200 ml/min total flow), then the standard will run out in 50 minutes. However, the calibration/match procedure was found to take 10 minutes to 15 minutes following initiation. Note that this time is recommended to fully replace the air in the tubing, water vapor traps, and IRGAs with the 400 ppm calibration air sample. Once CO₂ concentrations in each of the two IRGAs reaches steady state, record the offset from 400 ppm (should be less than 5 ppm) and initiate a point calibration of each IRGA with the stated concentration of 400 ppm (i.e., the CO₂ concentration in the standard).

Following each IRGA calibration, the two sample tubes can then be re-installed on the sample and ambient air inlets on the back of the case. The other end of the gas sample tubes are then connected to the outlet of the stem chamber (stem air sample) and inserted and secured in the ambient air reservoir (ambient air sample). A third ¼″ tube is also inserted and secured in the ambient air reservoir and connected to the ambient air inlet on the stem chamber.

CO₂ efflux measurements are then initiated by recording average CO₂ concentrations every about 30 seconds to 60 seconds on both ambient air and stem air IRGAs. Once measurements are initiated, the monitor is switched off with the mini-PC continuing to collect CO₂ data. The case can then be closed and left for continuous operation until the water absorbent needs replacing (about 24 hours in warm humid environments, like tropical forests).

Following completion of the measurements the following day, once the case is re-opened, the data logging is stopped and stored files are transferred to a USB drive. Following this, the system is transported to the next tree to be studied, followed by a new match/calibration procedure as necessary. However, even after continuous measurements on 3 to 7 different tree species during one week, the 400 ppm calibration/match procedure showed a low drift of the IRGAs with the CO₂ offset determined by weekly calibrations<5 ppm.

Following data collection, the stem CO₂ efflux rates were determined from 15-minute averages of the ambient air and stem air CO₂ concentration time series. Stem CO₂ efflux rates (Eₛ, μmol m⁻² s⁻¹) every 15 minutes were calculated according to equation 1, where F is the flow rate of ambient air through the stem chamber (0.1 L min⁻¹), Δ CO₂ (ppm) is the difference in CO₂ concentration between the stem air and ambient air, and A is the enclosed stem area (9.95E-3 m² (15.3 cm×6.5 cm)).

$${E}_{S} ( μ mol {m}^{- 2} {s}^{- 1} ) = F \times \frac{1 min}{60 s} \times \frac{1 μ mol}{22.4 μ L} \times \frac{Δ {CO}_{2}}{A} \qquad ( 1 )$$

### Example—Validation of Lightweight, Portable, Waterproof, and Low Power Dynamic Stem Respiration System for Trees

Described below is a field test of the low cost, waterproof, and portable stem respiration system for continuous observations of tree stem CO₂ efflux from 3D printed dynamic stem gas exchange chambers. This system, which is enclosed in a waterproof case, includes two Infrared Gas Analyzers (IRGAs) that continuously measured CO₂ concentrations from the ambient air reservoir near the stem and air exiting the gas exchange stem chamber (e.g., installed at breast height; the breast height of a tree is 4.5 feet above the ground). Prior to installing the system on the stem in the field, the calibration and match procedure was conducted as described in the previous EXAMPLE.

Data was collected from an Ash tree, Fraxinus sp., in Colorado, USA during the summer to validate the new method for determining real time stem Eₛ rates. The Ash tree genus is widespread and grows across much of Europe, Asia, and North America. The tree was estimated at 10 meters in height with a 70 cm to 80 cm diameter. The study was conducted in a suburban neighborhood in Fort Collins, Colorado, USA. The site receives an average annual precipitation of 409 millimeters (mm) with a low of 10 mm in January and a high of 61 mm in May. The soil is an Acidic Haplustalfs series which consists of fine-loamy very deep, well-drained soils. Raw CO₂ concentration data from the ambient and stem air IRGAs was recorded in real-time with a 1-minute logging frequency on the mini-PC starting at 8:00 AM. One delimited text file for the ambient air and stem air CO₂ concentration time series data was downloaded at the end of the 24 hour experiment. In addition, air temperature, which largely determines the magnitude of plant transpiration though its strong influence over the vapor pressure deficit (VPD) was also obtained for relations with stem Eₛ data. Air temperature was collected roughly 5 miles away at the Fort Collins Weather Station. In addition, stem temperature measurements were taken manually with a hand-held thermal imaging system for comparison with air temperature. All CO₂ and temperature data were averaged every 15 minutes prior to plotting and correlation analysis.

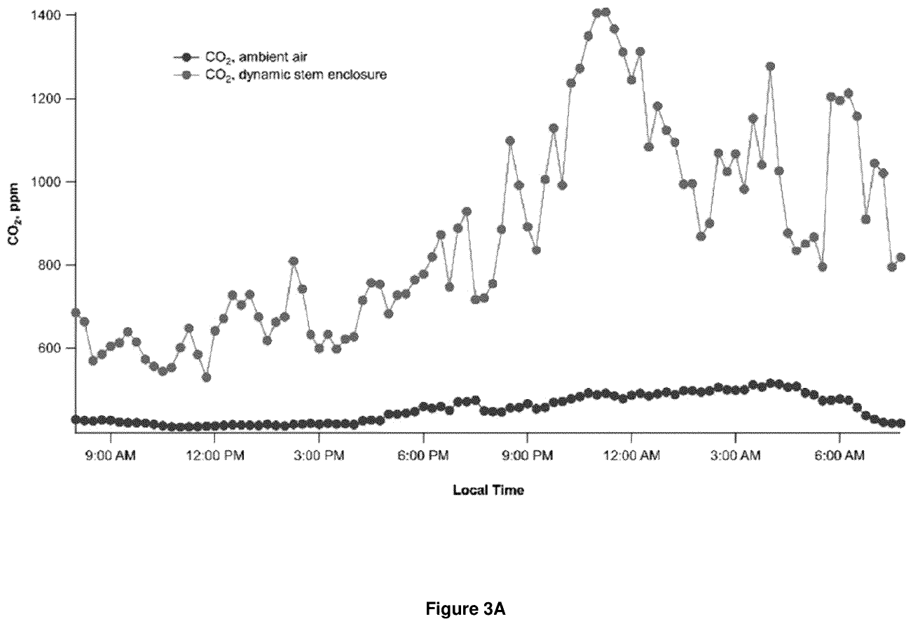

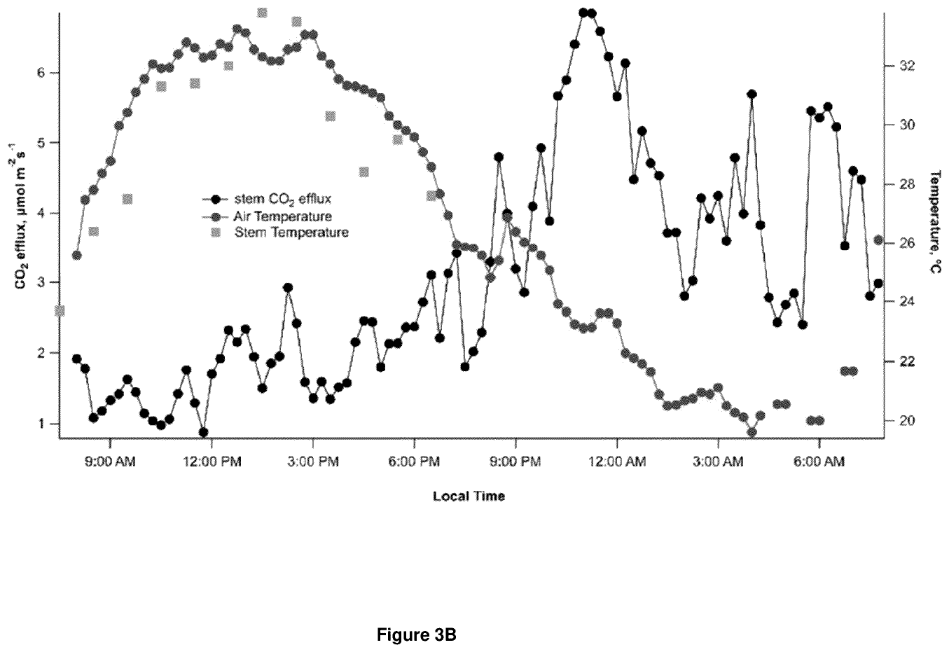

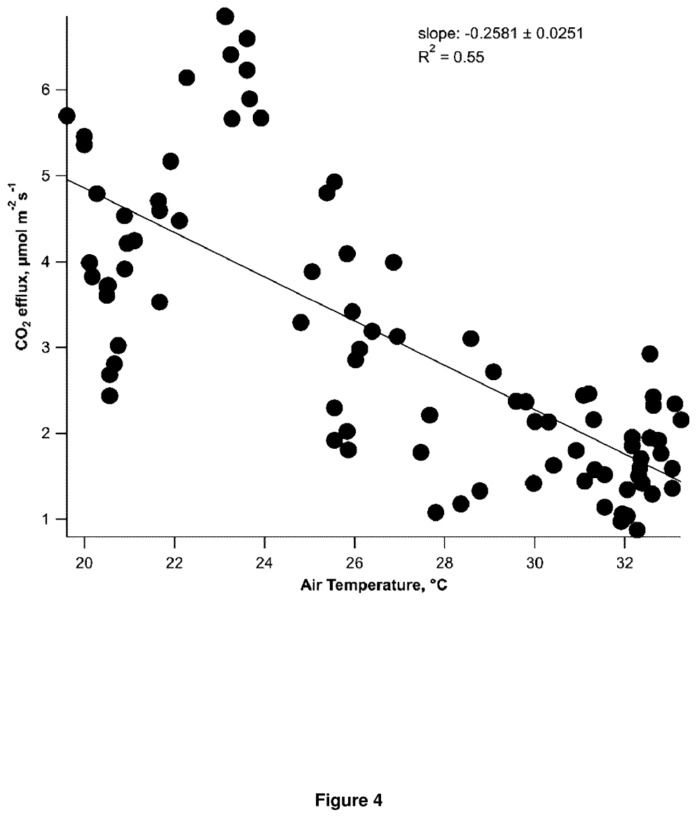

The results show that continuous positive gradient in CO₂ (Δ CO₂) was maintained by the stem emissions during both the day and night (FIG. 3A). Ambient air CO₂ varied throughout the 24-hour period reaching a maximum in the early morning pre-dawn period. Stem air CO₂ also varied substantially throughout the 24-hour period reaching a maximum near midnight. Ambient air CO₂ stayed at least 61 ppm below stem CO₂ at all times during the 24-hour period, with a maximum gradient occurring just prior to midnight. When Equation 1 was used to calculate the stem CO₂ efflux rates (Eₛ, μmol m⁻² s⁻¹) every 15 minutes, a diurnal trend was observed with Eₛ reaching higher values during the night and suppressed values during the day. Eₛ reached a maximum value of just prior to midnight of 6.8 μmol m⁻² s⁻¹ (FIG. 3B). In contrast, air temperature, and also likely tree transpiration, peaked around 2:00 PM in the afternoon. Moreover, when plotted versus air temperature, a negative relationship was observed with decreasing Eₛ with increasing temperature (FIG. 4). These observations are consistent with previous studies on diurnal Eₛ patterns of field trees, which showed a similar magnitude of Eₛ as well as a suppression during the daytime relative to the nighttime.

Although mitochondrial respiration is known to increase with temperature, recent studies have shown that daytime Eₛ is suppressed during the day relative to the night. However, the biological and physical mechanisms that give rise to Eₛ suppression is under discussion, and includes mechanisms like enhanced CO₂ storage, transport of CO₂ in the transpiration stream, suppression of stem mitochondrial respiration under reduced day-time stem turgor pressure, enhanced night-time growth rates, and stem CO₂ re-assimilation via both light dependent photosynthesis in green tissues and light-independent fixation via phosphoenylpyruvate carboxylase (PEP) as a part of anaplerotic metabolism. For example in a recent study, day-time Eₛ suppression was observed on young poplar trees growing in a greenhouse. This was attributed to temperature-dependent increases in xylem transport of locally respired CO₂ and lowered turgor pressure that constrained mitochondrial respiration. Thus, in order to verify daytime Eₛ suppression in other species, determine biological and environmental conditions where it does not occur, and discriminate between these mechanisms, the dynamic stem CO₂ efflux system presented herein will be valuable to the research community.

### Example—Experiments in Tropical Forests

In order to field test the portable dynamic stem CO₂ efflux system in a remote forested region of the world under heavy rain conditions, the system was deployed to Manaus, Brazil, during the rainy season. The results demonstrate that the system is capable of running off of a charged car battery for many weeks. Moreover, despite heavy rains in the remote field location, with the case closed and the system wrapped in a ground tarp, continuous CO₂ efflux observations were collected in diverse forest transects as well as remote locations. The system will be of use in tropical carbon cycle research with the goal of understanding the biological and environmental influences on diurnal and seasonal Eₛ patterns in diverse tropical forests.

### CONCLUSION

Further details regarding the embodiments described herein can be found in K. Jardine et al., “Development of a lightweight, portable, waterproof, and low power stem respiration system for trees,” MethodsX, Volume 10, 2023, 101986, which is hereby incorporated by reference.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A system comprising:
a chamber, the chamber including an inlet port and an outlet port, the chamber forming an enclosed volume when attached to a stem of a plant;
a first carbon dioxide sensor, the first carbon dioxide sensor including a first sensor inlet connected to the outlet port of the chamber; and
a pump connected to a first sensor outlet of the first carbon dioxide sensor, the pump operable to draw the air though the inlet port of the chamber and through the first carbon dioxide sensor.

2. The system of claim 1, wherein the chamber comprises a plastic.

3. The system of claim 1, wherein the chamber comprises polyethylene terephthalate glycol (PEG).

4. The system of claim 1, wherein the enclosed volume formed by the chamber and the stem of the plant is about 200 milliliters to 400 milliliters.

5. The system of claim 1, wherein an area on the stem of the plant forming the enclosed volume is about 90 cm² to 144 cm².

6. The system of claim 1, wherein dimensions of an area on the stem of the plan forming the enclosed volume are about 15 centimeters to 18 centimeters by about 6 centimeters to 8 centimeters.

7. The system of claim 1, wherein foam is position between the chamber and the stem of the plant.

8. The system of claim 1, wherein the first carbon dioxide sensor comprises a carbon dioxide infrared gas analyzer.

9. The system of claim 1, further comprising:
a power source operable to power the first carbon dioxide sensor and the pump.

10. The system of claim 1, further comprising:
a computing device operable to record carbon dioxide concentration information output from the first carbon dioxide sensor.

11. The system of claim 1, further comprising:
a buffer volume, wherein a first tube inlet of a first tube is positioned in the buffer volume, and wherein a first tube outlet of the first tube is connected to the inlet of the chamber.

12. The system of claim 1, further comprising:
a water trap positioned between the chamber and the first carbon dioxide sensor.

13. The system of claim 12, wherein the water trap comprises a membrane dryer.

14. The system of claim 12, wherein the water trap includes clay granules and dolomite.

15. The system of claim 1, further comprising:
a second carbon dioxide sensor, wherein the second carbon dioxide sensor includes a second sensor inlet, wherein the second sensor inlet is operable to sample ambient air, wherein the pump is connected to a second sensor outlet of the second carbon dioxide sensor, and wherein the pump is further operable to draw the ambient air though the second sensor inlet and through the second carbon dioxide sensor.

16. The system of claim 15, wherein the second carbon dioxide sensor comprises a carbon dioxide infrared gas analyzer.

17. The system of claim 15, further comprising:
a second water trap positioned between the second sensor inlet and the second carbon dioxide sensor.

18. The system of claim 15, wherein the computing device is further operable to record carbon dioxide concentration information output from the second carbon dioxide sensor.

19. The system of claim 15, further comprising:
a buffer volume, wherein a first tube inlet of a first tube and a second tube inlet of a second tube are positioned in the buffer volume, wherein a first tube outlet of the first tube is connected to the inlet of the chamber, and wherein a second tube outlet of the second tube is connected to the second sensor inlet of the second carbon dioxide sensor.

20. A system comprising:
a chamber, the chamber including an inlet port and an outlet port, the chamber forming an enclosed volume when attached to a stem of a plant;
a first carbon dioxide sensor, the first carbon dioxide sensor including a first sensor inlet connected to the outlet port of the chamber;
a second carbon dioxide sensor, the second carbon dioxide sensor including a second sensor inlet, the second sensor inlet operable to sample ambient air; and
a pump connected to a first sensor outlet of the first carbon dioxide sensor and a second sensor outlet of the second carbon dioxide sensor, the pump operable to draw the air though the inlet port of the chamber and through the first carbon dioxide sensor, and the pump operable to draw the ambient air though the second sensor inlet and through the second carbon dioxide sensor.
